(19) United States
(12) Patent Application Publication
(10) Pub. No.: US 2016/0270663 A1
(43) Pub. Date: Sep. 22, 2016

(54) FLUORESCENT IMAGE ACQUISITION DEVICE

(71) Applicant: SHIMADZU CORPORATION, Kyoto (JP)

(72) Inventor: Tatsuya IKEHARA, Kyoto (JP)

(73) Assignee: Shimadzu Corporation, Kyoto (JP)

(21) Appl. No.: 14/916,238

(22) PCT Filed: Sep. 10, 2013
(86) PCT No.: PCT/JP2013/074380

§ 371 Date: Jun. 6, 2016

(51) Int. Cl. A61B 5/00; G01N 21/64

(52) U.S. Cl. CPC A61B 5/0071; A61B 5/0042; G01N 21/6486; A61B 5/742; A61B 5/7242; A61B 5/7475; G01N 21/6456

(57) ABSTRACT

A method for acquiring a fluorescent image including a step of irradiating a sample with excitation light from a position distanced from the sample to generate fluorescence, and detecting the generated fluorescence by a two-dimensional detector to obtain a fluorescent image; at least one step of irradiating the sample with excitation light in the same manner in the condition that entry of fluorescence from a part emitting the strongest fluorescence at the time when fluorescence is to be detected is substantially blocked, to obtain a fluorescent image by the two-dimensional detector; a step of integrating at least two fluorescent images including the fluorescent image obtained in the step and at least one fluorescent image obtained in the step into one image; and a step of displaying the image integrated in the step.

### TECHNICAL FIELD

The present invention relates to a device and a method for acquiring a fluorescent substance in a sample as an image, and for example, relates to a device and a method for acquiring a fluorescent image inside a living body directed at living body samples of small animals and the like.

### BACKGROUND ART

Fluorescent image acquisition devices can be roughly classified into two systems.

One system is (A) a scanning fluorescent image acquisition device. In this device, sufficiently collected excitation light is radiated from one point of a sample to be observed to excite a fluorescent substance existing in the sample, fluorescence generated from the fluorescent substance is received, and a fluorescent image is acquired. By scanning so that the radiation points cover the whole area, a fluorescent image of the entire sample is obtained (see Patent Documents 1, 2). Also known is a device having a tomographic imaging function of estimating the position of the fluorescent substance from each obtained fluorescent image data.

One feature of the scanning fluorescent image acquisition device is that an observation area can be designated. For example, when a brain of a living body sample is intended to be observed, it is possible to remove the background fluorescence from areas other than the brain, and to obtain a sufficient dynamic range by setting only the brain area as a scanning point. However, in in vivo observation, such background fluorescence can have important information. For example, in conducting a drug metabolism evaluation, the drug, if accumulated in locations other than the target area, can influence as a side effect of the drug. For this reason, it is first necessary to scan the whole living body; however, a very long time is required for scanning the whole area of the object to be observed in the scanning device.

Another system is (B) an all-round radiation type fluorescence acquisition device. In this device, a wide area in a sample is simultaneously irradiated with excitation light to obtain a fluorescent image of the wide area at once (see Patent Document 3). Also known is a device having a tomographic imaging function of estimating the position of the fluorescent substance from each obtained fluorescent image data, although the number of pieces of data is small.

One feature of the all-round radiation type fluorescence acquisition device is that a fluorescent image of the whole area of the object to be observed can be acquired in a short time. This feature overcomes the defect of the scanning type. For example, in the application directed at drug screening with the use of mice or monitoring during an operation, it is expected that a fluorescent can be obtained image in a short time. However, in the whole area observation, there is a case that a sufficient dynamic range cannot be obtained due to the influence of background fluorescence. For example, in evaluation of antibody pharmaceuticals, there is a case that faint fluorescence in the target area cannot be detected due to accumulation in the area other than the objective target (liver accumulation, in particular). For example, in the case of lymph node observation utilizing ICG (Indo Cyanine Green), since the fluorescent intensity is very high in the part where ICG is injected, there is a case that faint fluorescence of the lymph node terminal cannot be detected.

For this reason, a method of widening the dynamic range by providing (C) all-round radiation type with an intensity distribution of excitation light is proposed (see Patent Document 4). Although this method is effective when the depth direction of the fluorescent substance is not taken into account (DNA detection or the like), it is necessary to vary the excitation condition depending on the depth direction of the medium in somatometry of a mouse, a human being, or the like, and hence, the method of (C) cannot be simply diverted. Also, even if the excitation light is weakened depending on the excited part, the fluorescent intensity would be large if the amount of the fluorescent substance in that part is large, and hence, some handling mechanism is required on the fluorescence detection side.

Although it is conceivable to attach a mechanism that will not cause halation to a fluorescence two-dimensional detector, a problem arises in the quantitative analysis because linearity of the fluorescent intensity cannot be ensured in this case.

### PRIOR ART DOCUMENTS

### Patent Documents

Patent Document 1: U.S. Pat. No. 6,615,063

Patent Document 2: US 2012/0017842 A

Patent Document 3: U.S. Pat. No. 8,345,941

Patent Document 4: U.S. Pat. No. 7,307,261

Patent Document 5: US 2011/0199500 A

### SUMMARY OF THE INVENTION

### Problems to be Solved by the Invention

As described above, the scanning type device has a problem that the observation time is extremely long although it can ensure a wide dynamic range. On the other hand, the all-round radiation type device has a problem that the dynamic range is narrow and relatively faint fluorescence cannot be detected although a short time and continuous time observation is possible.

The present invention includes not only the case of acquiring a fluorescent image of a living body, but also the case of acquiring a fluorescent image of a sample other than living bodies, and has an object of acquiring an image of a fluorescent substance of the whole area to be observed efficiently in a wide dynamic range.

### Solution to the Problems

A fluorescent image acquisition device of the present invention includes a sample stage, an excitation light source for irradiating a sample placed on the sample stage with excitation light which is a spreading light flux from a position distanced from the sample to excite a fluorescent substance in the sample to generate fluorescence for making the irradiation of the sample with the excitation light into an all-round radiation type, an image pickup unit having a two-dimensional detector for detecting fluorescence from the sample, and a display unit for displaying an image based on a fluorescent image acquired by the image pickup unit.

The fluorescent image acquisition device of the present invention further includes a blocking mechanism disposed between the sample and the two-dimensional detector, for blocking entry of part of the fluorescence from the sample into the two-dimensional detector; a blocking control unit for controlling the blocking mechanism in such a manner that entry of the strongest fluorescence from the sample into the two-dimensional detector is substantially blocked at a time when an image is to be taken by the image pickup unit; and an image integrating unit for calculating and processing in such a manner that at least two fluorescent images including at least one fluorescent image obtained by the image pickup unit in the condition that part of the sample is blocked by the blocking mechanism are integrated into one image. The display unit is configured to display an integrated image obtained by the image integrating unit.

The wording “substantially block” in substantially blocking the strongest fluorescence entering the two-dimensional detector by the blocking control unit means blocking the strongest fluorescence to such a degree that the second strongest fluorescence after the strongest fluorescence can be detected. As one example of substantially blocking, the intensity of the strongest fluorescence is attenuated to about one-fifth so as to enable detection of faint fluorescence by increasing the dynamic range of the area other than the blocking part to five times in the example later described by referring to FIG. 7. Attenuating to about one-fifth in that case is equivalent to substantially blocking. The attenuation rate is not limited in particular, and may be larger or smaller than one-fifth. As a matter of course, the case of blocking 100% is also included.

A method for acquiring a fluorescent image of the present invention includes the following steps (A) to (D).

(A) a step of irradiating a sample with excitation light which is a spreading light flux from a position distanced from the sample to excite a fluorescent substance in the sample to generate fluorescence, and detecting the generated fluorescence by a two-dimensional detector to obtain a fluorescent image;

(B) at least one step of irradiating the sample with excitation light in a same manner as in the step (A) in a condition that entry of fluorescence from a part emitting the strongest fluorescence at a time when fluorescence is to be detected by the two-dimensional detector into the two-dimensional detector is substantially blocked, to obtain a fluorescent image by the two-dimensional detector;

(C) a step of integrating at least two fluorescent images including the fluorescent image obtained in the step (A) and at least the one fluorescent image obtained in the step (B) into one image; and

(D) a step of displaying the image integrated in the step (C).

In the step (A), a fluorescent image in the whole area to be observed of the sample is acquired. The whole area to be observed may be the entire sample, or part of the sample. In this case, fluorescence from a fluorescent substance in the sample is detected depending on the dynamic range of the two-dimensional detector.

In the step (B), the blocking mechanism for physically blocking fluorescence is actuated and the strongest fluorescence from the sample is substantially blocked based on positional information of the fluorescent substance in the interior of the sample. The positional information of the fluorescent substance in the interior of the sample can be obtained, for example, by the calculation processing unit for determining living body internal information from a fluorescent image, or based on input sample internal information. For blocking the strongest fluorescence, the excitation light may be blocked so that the fluorescent substance generating the strongest fluorescence is not irradiated with the excitation light, or the fluorescence may be blocked so that the fluorescence does not enter the two-dimensional detector even if the fluorescent substance generating the strongest fluorescence is irradiated with the excitation light and the fluorescence is generated. Further, a fluorescent image is acquired in the condition that the strongest fluorescence from the sample at the time of image pickup is substantially blocked. At this time, since the fluorescence from the strongest fluorescent substance is substantially blocked, fluorescence from the fluorescent substance having the second strongest fluorescent intensity is detected. This detection is conducted with higher sensitivity compared with the image pickup condition in the step (A), for example, by elongating the exposure time of the two-dimensional detector.

Further, the processing of the step (B) is repeated as needed. In the second-time step (B), since the fluorescence detected in the step (A) is already blocked substantially, the fluorescence detected in the first-time step (B) is the strongest fluorescence in the second-time step (B). Detection of the second-time step (B) is conducted with higher sensitivity in comparison with the image pickup condition of the first-time step (B), for example, by elongating the exposure time of the two-dimensional detector. The same applies in conducting the third—or later time step (B).

By varying the image pickup condition (exposure time or the like) of the two-dimensional detector for each step, it is possible to eventually extend the dynamic range, and it becomes possible to detect fluorescence while ensuring the linearity from strong fluorescence to faint fluorescence. Since the processing including the step (A) and the step (B) is repeated about two to five times at most, a shorter time is required compared with the case of taking image of the whole area by a scanning type device, and a dramatically effective process is achieved.

### Effects of the Invention

The present invention is featured by integrating at least two fluorescent images including a fluorescent image obtained by the image pickup unit in the condition that entry of the strongest fluorescence from the sample at the time of image pickup into the two-dimensional detector is substantially blocked into one image. As a result, it is possible to detect fluorescence of the whole area to be observed in a wider dynamic range compared with the all-round radiation type device, and to detect fluorescence of the whole area to be observed in a shorter time compared with the scanning type device, and hence, the efficiency is improved. It is particularly useful, for example, when the fluorescent substance is accumulated in a plurality of sites in the living body, or when faint fluorescence that is often missed under the cover of strong fluorescence is detected.

### BRIEF DESCRIPTION OF THE DRAWINGS

FIG. 1 is a block diagram showing one embodiment of a fluorescent image acquisition device.

FIG. 2 is a flowchart showing one example of operation of one embodiment.

FIG. 3 is a schematic diagram showing one example of operation of one embodiment, in which FIG. 3(A) shows the condition of acquiring a fluorescent image in the whole area to be observed, and FIG. 3(B) shows the condition of acquiring a fluorescent image in the condition that fluorescence from a specific location is blocked so that it does not substantially enter the two-dimensional detector.

FIG. 4 is a view showing a first specific example of a blocking mechanism.

FIG. 5 is a view showing a second specific example of a blocking mechanism.

FIG. 6 is a diagram illustrating a method of determining the range to be blocked by the blocking mechanism, in which FIG. 6(A) is a section view showing a fluorescent substance disposed in a living body, and fluorescence, and FIG. 6(B) shows a profile of fluorescent intensity detected on the surface of a living body sample.

FIG. 7 is a chart showing a profile of fluorescent intensity obtained by normalizing the profile of FIG. 6(B) so that the fluorescent intensity at a distance of 20 mm is 1.

FIG. 8 is a flowchart showing one example of operation of the blocking mechanism shown in FIG. 4.

FIG. 9 is a flowchart showing one example of operation of the blocking mechanism shown in FIG. 5.

FIG. 10 is a view showing fluorescence detection of the whole area to be observed of the cylindrical phantom, in which FIG. 10(A) shows the phantom and a tube containing a fluorescent substance embedded therein, and FIG. 10(B) shows an image showing a fluorescent image of the whole area to be observed.

FIG. 11 is a longitudinal section view (left) and a cross section view (right) showing a fluorescent image reconfigured based on the fluorescent image data of FIG. 10(B).

FIG. 12 is a view showing fluorescence detection in the condition that the cylindrical phantom is partly blocked, in which FIG. 12(A) is a perspective view showing a phantom and a blocking mechanism covering part of the same, and FIG. 12(B) is an image showing a detected fluorescent image.

FIG. 13 is a longitudinal section view (left) and a cross section view (right) showing a fluorescent image reconfigured based on the fluorescent image data of FIG. 12(B).

FIG. 14 is a view showing a method of displaying the fluorescent images in FIGS. 10 and 12 on the same screen in different two colors.

FIG. 15 is a view showing a method of displaying the fluorescent images in FIGS. 11 and 13 on the same screen in different two colors.

### EMBODIMENTS OF THE INVENTION

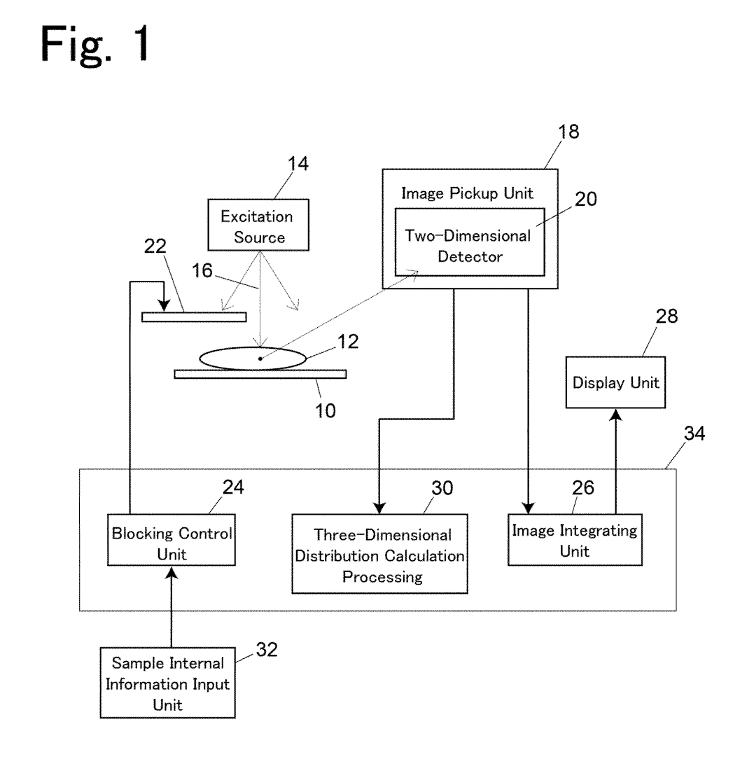

One embodiment of a fluorescent image acquisition device is shown in FIG. 1.

On a sample stage 10, a sample 12 to be measured is placed. Non-limiting examples of the sample 12 include living body samples like small animals, such as a mouse. An excitation light source 14 has an excitation light source that irradiates the sample 12 placed on the sample stage 10 with excitation light 16 which is a spreading light flux from a position distanced from the sample 12 to excite a fluorescent substance in the sample 12 to generate fluorescence. As the excitation light source, an LD (laser diode) or an LED (light emitting diode) that emits excitation light of the required excitation wavelength, and other lamps such as a halogen lamp can be used. In the excitation light source 14, it is preferred to use an excitation side interference filter for removing the light of the fluorescent wavelength to be detected in combination with the excitation light source.

When the excitation light source 14 has a three-dimensional distribution calculation processing unit as a preferred embodiment, an illuminating light source such as a white LED for illuminating a sample and taking an appearance image of the sample is also provided in order to obtain three-dimensional surface profile data for use in the direct problem analytical processing and the inverse problem analytical processing from the taken appearance image.

The image pickup unit 18 has a two-dimensional detector for detecting fluorescence from the sample 12. The two-dimensional detector 20 is, for example, a CCD (charge coupled device). The image pickup unit 18 also includes an optical system such as a condensing lens for guiding the fluorescence from the sample 12 to the two-dimensional detector 20, and a fluorescence side interference filter for removing the excitation light component and allowing permeation of the fluorescence to be detected in order to prevent the excitation light component from entering the two-dimensional detector 20.

A blocking mechanism 22 is provided for preventing the fluorescence from a predetermined position of the sample 12 from entering the two-dimensional detector 20. While specific examples of the blocking mechanism 22 will be described later, a mechanism that automatically operates by the electric control from a blocking control unit 24 is preferably provided. The blocking control unit 24 controls the blocking mechanism 22 so that the blocking mechanism 22 substantially blocks entry of the strongest fluorescence from the sample 12 into the two-dimensional detector 20 at the time of image pickup.

An image integrating unit 26 performs a calculation process of integrating at least two fluorescent images including the fluorescent image obtained by the image pickup unit 18 in the condition that apart of the sample 12 is blocked by the blocking mechanism 22 into one image. A display unit displays the integrated image obtained by the image integrating unit 26. Non-limiting examples of the display unit 28 include a liquid crystal display. Preferably, the display unit 28 is configured to display the integrated image in different colors for different intensity ranges.

The blocking control unit 24 controls the blocking mechanism 22 so that entry of the strongest fluorescence from the sample 12 at the time of image pickup into the two-dimensional detector 20 is substantially blocked based on the sample internal information. For obtaining the sample internal information for the control, a three-dimensional distribution calculation processing unit 30 for obtaining sample internal information from the fluorescent image data obtained by the image pickup unit 18 is provided in one embodiment. In that case, the blocking control unit 24 controls the blocking mechanism 22 based on the sample internal information obtained by the three-dimensional distribution calculation processing unit 30.

For obtaining the sample internal information required by the blocking control unit 24 to control the blocking mechanism 22, a sample internal information input unit 32 for inputting internal information of a sample to be detected is further provided in other embodiment. In that case, the blocking control unit 24 controls the blocking mechanism 22 based on the sample internal information input from the input unit 32.

When the three-dimensional distribution calculation processing unit 30 is provided, the image integrating unit 26 may be configured to integrate the fluorescent image reconfigured by the three-dimensional distribution calculation processing unit 30.

The blocking control unit 24, the image integrating unit 26, and the three-dimensional distribution calculation processing unit 30 are implemented by a computer 34. The computer 34 is a computer dedicated to the fluorescent image acquisition device or a personal computer. The input unit 32 is an input device including a keyboard and a mouse for inputting data to the computer 34.

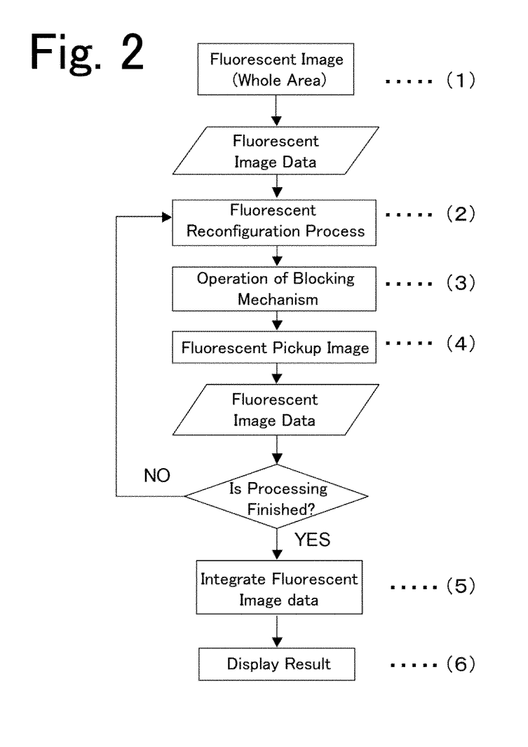

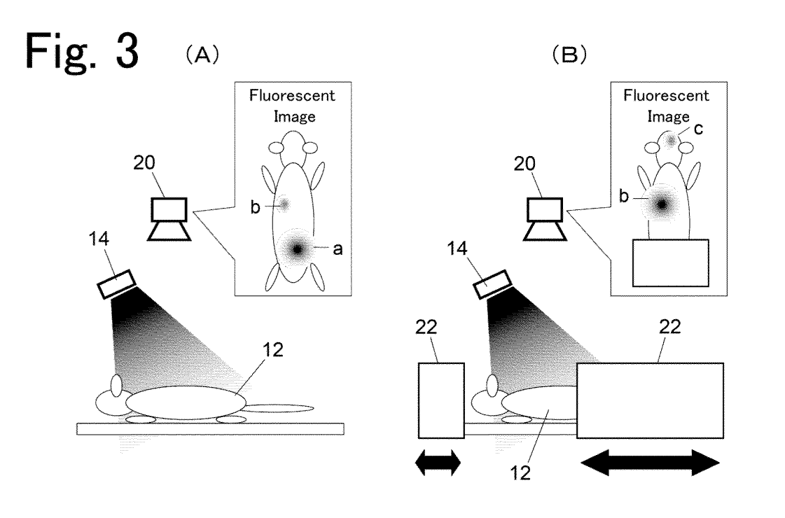

Referring to FIG. 2 and FIG. 3, one exemplary operation of one embodiment will be described.

### (Procedure 1) (FIG. 2 (1))

First of all, the whole area to be observed of the sample is irradiated with the excitation light 16 from the excitation light source 14, and a fluorescent image in the whole area to be observed is acquired by the two-dimensional detector 20 of the image pickup unit 18 (FIG. 3(A)). The whole area to be observed is not necessarily the entire sample, and may be a part to be observed in the sample. Non-limiting examples of the sample include living body samples. In this process, the site where the fluorescent intensity is highest is detected with high sensitivity. In the example of FIG. 3(A), the site designated by the mark “a” is the site where the fluorescent intensity is highest, and fluorescence from that site is detected with high sensitivity. Meanwhile when one intends to take an image also from faint fluorescence of other site b with high sensitivity in the condition for detecting fluorescence from the site a, it is necessary to raise the detection sensitivity of the two-dimensional detector, accordingly, the dynamic range of the two-dimensional detector is insufficient, and halation occurs due to the strong fluorescence from the site a.

### (Procedure 2) (FIG. 2 (2))

Next, based on the fluorescent image data obtained in the aforementioned process, living body internal information is determined by the three-dimensional distribution calculation processing unit 30. Specific examples of the processing procedure for determining living body internal information include a method of calculating a tomogram by the direct problem analysis and the inverse problem analysis, and a method of deriving deep part information from different wavelengths (see Patent Document 5). When the accumulation site is previously known as in subcutaneous blood vessels and lymph nodes, it is also possible to input living body internal information from the sample internal information input unit 32 in place of determining living body internal information by the three-dimensional distribution calculation processing unit 30.

Hereinafter, as one example of the processing procedure for determining living body internal information, the processing procedure of the method of calculating a tomogram by the direct problem analysis and the inverse problem analysis will be described. The direct problem analysis is established from the process in which excitation light travels inside the living body when the excitation light is emitted to the living body sample from an arbitrary direction, and the process in which the fluorescence travels inside the living body when a fluorescent substance exists in an arbitrary position inside the living body. The respective processes can be calculated by the light diffusion equations (1) and (2) shown below.

−∇D(r)∇φₑₓ₍ⱼ₎(r)+μₐ(r)φₑₓ₍ⱼ₎(r)=S₍ⱼ₎(r) (1)

−∇D(r)∇φₑₘ₍ₖ₎(r)+μₐ(r)φₑₘ₍ₖ₎(r)=εγMφₑₓ₍ⱼ₎(r) (2)

Here, the above symbols respectively designate:

D: diffusion constant,

μa: absorption coefficient,

φex(j): excitation light fluence rate at position r when the excitation light is given from the j-th direction

φem(k): fluorescence fluence rate at position r when a fluorescent substance exists at the k-th position,

ε: molar absorption coefficient,

γ: quantum yield,

M: molar concentration, and

S(j): excitation light intensity at the j-th position.

From the formula (1) and the formula (2), theoretical values of excitation and fluorescence transmission can be determined, and based on the calculation results, system matrix A is prepared. System matrix A is a matrix for obtaining a theoretical value of fluorescence distribution detected on the surface of the living body when spatial distribution f of the fluorescent substance is given. Specifically, column vector v of the system matrix is a theoretical value of fluorescence distribution detected on the surface of the living body at an arbitrary position, and the column vectors are determined up to the k-th position to give the system matrix.

g=Af (3)

A=[v(1), v(2), . . . , v(k)] (4)

Here, the above symbols respectively designate:

A: system matrix,

f: spatial distribution vector of fluorescent substance, and

g: fluorescence distribution vector on the living body surface. For example, when the spatial distribution of fluorescent substance f=[0 0 0.5 1 0.5 0 . . . 0]⁷ is given, a fluorescence distribution vector on the living body surface is obtained by multiplying the vector with the system matrix (Af). The [A B C]^{T} is a transposed matrix.

The operation in the inverse problem analysis is established by the process of determining a spatial distribution of a fluorescent substance from fluorescence detection data obtained in the device, and a theoretical value calculated by the direct problem analysis. First, system matrix A in the formula (3) can be determined by the direct problem analysis. Meanwhile, fluorescence distribution vector g on the living body surface in the formula (4) can be obtained by a fluorescence living body image acquisition device. In the inverse problem analysis, from system matrix A determined by the direct problem analysis and fluorescence distribution vector g on the living body surface, spatial distribution vector f of an unknown fluorescent substance is determined. A common method for determining vector f is the least squares method, and vector f can be determined by minimizing the evaluation function of the formula (5) below.

K(f)=∥g−Af∥² (5)

On the other hand, in the problem focused on in this case, since influence of the measurement noise is strong by the absorption and scattering of the light, it is highly possible that the solution diverges in the minimization problem of the formula (5). For the purpose of preventing the divergence of the noise, the minimization problem of the following formula (6) is considered. The minimization problem (Tikhonov regularization) is commonly used in the field of study of inverse problems, and divergence of the solution can be prevented by incorporating the norm of vector f into the evaluation function.

K(f)=∥g−Af∥²+λ∥f∥² (6)

Here, λ represents a normalization parameter. The living body internal information can be determined in this manner, and an image of the fluorescent substance inside the living body can be reconfigured.

### (Procedure 3) (FIG. 2 (3))

Since the positional information inside the living body at the site a where the fluorescent substance exists is obtained in Procedure 2, the blocking control unit 24 actuates the blocking mechanism 22 for physically blocking the fluorescence for the site a based on the information, to remove the fluorescence from the site a (FIG. 3B). For blocking the fluorescence from the site a so that it does not enter the two-dimensional detector 20, part of the excitation light is blocked by the blocking mechanism 22 so that the excitation light does not enter the fluorescent substance at the site a, or the fluorescence is blocked by the blocking mechanism 22 so that the fluorescence generated from the fluorescent substance at the site a does not enter the two-dimensional detector 20 even if the excitation light enters the fluorescent substance at the site a.

### (Procedure 4) (FIG. 2 (4))

In Procedure 3, for the observation range for which entry of the fluorescence into the two-dimensional detector 20 from the site a is substantially limited, a fluorescent image is acquired again (FIG. 3B). At this time, the image is taken while the acquisition condition of the two-dimensional detector 20 is varied so that the fluorescence from the site b can be sufficiently detected.

Further, when fainter fluorescence as designated by the symbol c in FIG. 3B is detected, Procedures 2 to 4 are repeated to detect the fluorescent image thereof. The same applies in detecting further fainter fluorescence.

### (Procedure 5) (FIG. 2 (5))

For the obtained plurality of pieces of fluorescent image data, the image data is integrated. In integrating the image data, the image data is integrated for the same acquisition condition of the two-dimensional detector 20. The image data to be integrated may be fluorescent image data acquired by the two-dimensional detector 20, or may be fluorescent image data reconfigured in Procedure 2.

### (Procedure 6) (FIG. 2 (6))

The data obtained in Procedure 5 is displayed on the display unit 28. The dynamic range of the display unit 28 can be insufficient at the time of display even though measurement with an extended dynamic range is possible at the time of observation. For this reason, the integrated image is displayed in any of the following manners: (a) displaying while the displayed range is arbitrarily set, (b) displaying logarithmically, and (c) displaying in different colors while the fluorescent intensity is divided into several ranges.

Next, a specific example of the fluorescence blocking mechanism 22 is described.

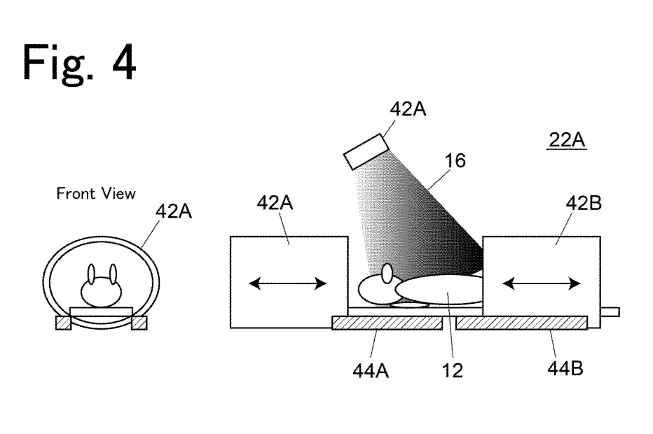

FIG. 4 is an example of a blocking mechanism 22A disposed around a sample of a small living body such as a mouse. Cylindrical blocking plates 42A, 42B having a diameter of about 70 mm are disposed in front and back, two positions, in the longitudinal direction of the living body sample 12. The longitudinal direction of the living body sample 12 refers to the direction of the straight line connecting the head part to the tail part of the living body. The blocking plates 42A, 42B are supported by respective electric sliders 44A, 44B so that they can move along the longitudinal direction of the living body sample 12. The blocking plates 42A, 42B are preferably made of light materials such as aluminum or plastic, and the surfaces thereof are preferably painted in black to minimize the light reflex. The blocking plates 42A, 42B are driven by the electric sliders 44A, 44B by the instruction from the blocking control unit 24, to move to an arbitrary area to block the light.

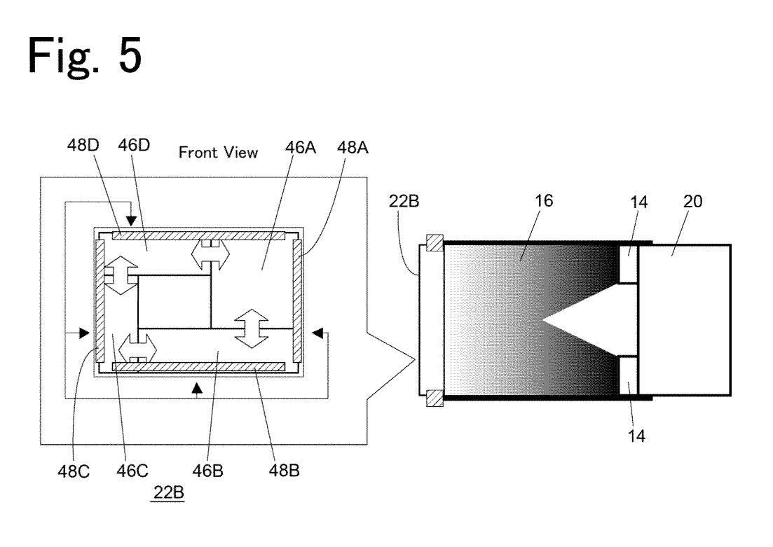

FIG. 5 is an example of a blocking mechanism 22B disposed between the excitation light source 14 and the two-dimensional fluorescence detector 20, and the sample 12. In this example, two excitation light sources 14 are disposed in front of the two-dimensional fluorescence detector 20. The number of the excitation light source 14 may be one, or may be three or more. The excitation light source 14 irradiates the sample (not illustrated) with the excitation light 16 over a wide angle. The blocking mechanism 22B is disposed between the positions where the excitation light source 14 and the two-dimensional fluorescence detector 20 are disposed, and the sample. The blocking mechanism 22B blocks radiation of part of the excitation light 16 from the excitation light source 14 to the sample, and blocks entry of part of the fluorescence from the sample into the two-dimensional fluorescence detector 20.

The blocking mechanism 22B has four blocking plates 46A to 46D disposed in the same plane, and the blocking plates 46A to 46D are supported so that they move independently by respective electric sliders 48A to 48D. The blocking plates 46A to 46D are preferably made of light materials such as aluminum or plastic, and the surfaces thereof are preferably painted in black to minimize the light reflex. The blocking plates 46A to 46D are driven by the electric sliders 48A to 48D by the instruction from the blocking control unit 24, to move to an arbitrary area to block the light.

It is preferred that the blocking mechanism for physically blocking excitation and fluorescence is actuated in correspondence with the positional information of the fluorescent substance inside the living body. At this time, for example, when a fluorescent substance that emits the strongest fluorescence is present at a shallow position inside the living body viewed from the two-dimensional detector 20, the influence of scattering when the fluorescence permeates inside the living body is small, so that the blocking range for removing the fluorescence can be narrowed. On the other hand, when the fluorescent substance is present at a deep position inside the living body viewed from the two-dimensional detector 20, the influence of scattering when the fluorescence permeates inside the living body is large, so that it is necessary to extend the blocking range for removing the fluorescence. In this manner, it is possible to provide an optimum image pickup condition while taking the scattering problem which is problematic in somatometry into account.

The range to be blocked by the blocking mechanism 22 is determined, for example, in the following manner.

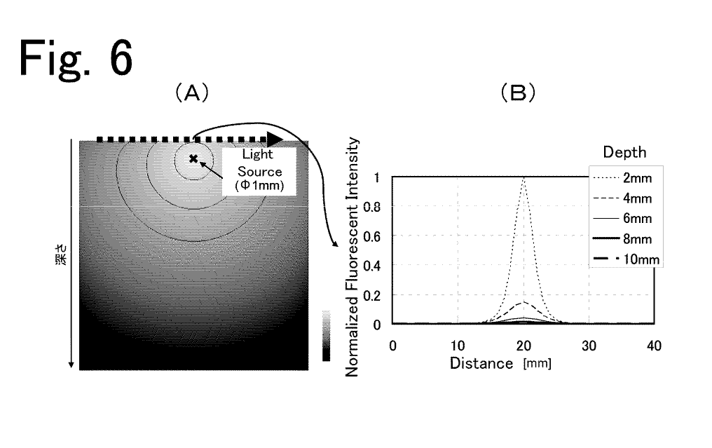

First of all, a simulation is conducted to find to what degree the fluorescence emitted from the fluorescent substance inside the living body spreads when it is detected on the living body sample surface. The example is illustrated in FIG. 6. As illustrated in FIG. 6(A), a light source having a diameter of 1 mm is disposed inside the living body (absorption coefficient 0.02 [/mm], scattering coefficient 10 [/mm]). FIG. 6(B) shows a profile of fluorescent intensity detected on the living body sample surface when the depth of disposition is varied. The profile can be calculated by the light diffusion equations (1) and (2).

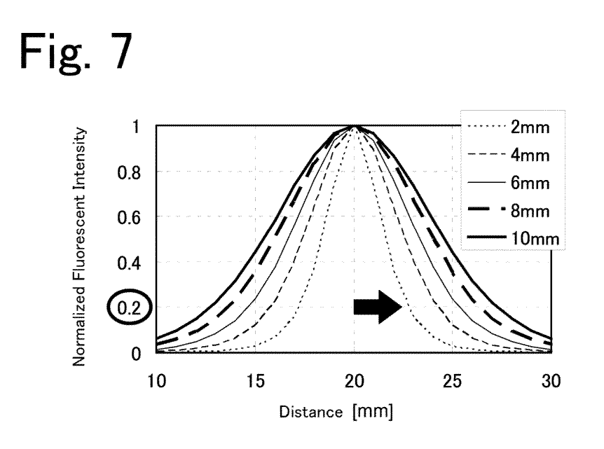

FIG. 7 shows a profile of fluorescent intensity normalized from FIG. 6 (B) so that the fluorescent intensity at a distance of 20 mm is 1. From FIG. 7, it is possible to obtain an index regarding to what degree the fluorescence inside the living body sample spreads when it is detected on the surface. The range in which fluorescence is to be blocked is determined based on the information obtained from FIG. 7. For example, when the fluorescent substance is present in the position of mm deep of the living body sample viewed from the two-dimensional detector 20, it is possible to attenuate the fluorescence coming from the fluorescent substance to about one-fifth by blocking the range of about 3 mm from the peak point of the fluorescent intensity. With this blocking, it becomes possible to increase the dynamic range of the area other than the blocked part to about five times, and it becomes possible to detect even faint light. The one-fifth attenuation indicates one example of substantial blocking.

By preparing a table in which the positional information and the blocking range are brought into correspondence with each other based on the finding obtained from FIG. 7 in advance, and storing the table in the computer 34, the blocking control unit 24 can designate the blocking range efficiently. When the accumulation site of the fluorescent substance is previously known as in various organs such as liver or subcutaneous blood vessels and lymph nodes, the positional information of the fluorescent substance inside the living body sample is also known in advance, and hence, the range to be blocked can be preliminarily input from the input unit 32 and stored in the computer 34 after once conducting the simulation as illustrated in FIG. 7.

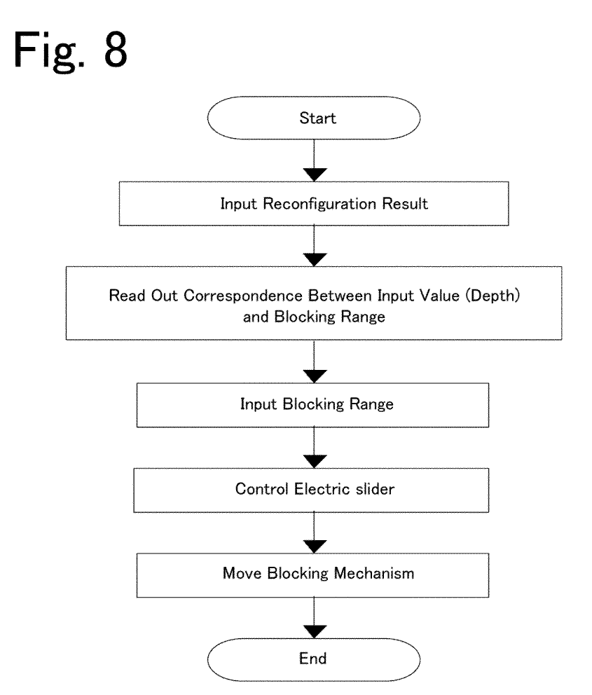

Hereinafter, one example of the operation of the blocking mechanism is shown. The case where the blocking mechanism 22 is the blocking mechanism 22A shown in FIG. 4 will be described by referring to FIG. 8. The blocking mechanism 22A is suited for the case where the sample to be measured is a small living body such as a mouse. The computer 34 stores a correspondence table between the depth and the blocking range. One example of the correspondence table is shown in Table 1.

TABLE 1

| | |
|---|---|
| 1 | Depth (mm) |
| 2 | Blocking range (mm) (radius from fluorescent intensity |
| | peak point) |

The correspondence table is preliminarily determined by calculation so that the fluorescence entering the two-dimensional detector 20 is attenuated to one-fifth by blocking. The degree of light attenuation of the fluorescence entering the two-dimensional detector 20 is a degree for substantially blocking so that the fluorescence having the second highest intensity after that fluorescence can be detected, and can be varied as needed.

As a precondition, the first-time image pickup of detecting a fluorescent image from the whole area of the object to be observed without using the blocking mechanism 22A has already been carried out.

For the second-time fluorescent image pickup, the blocking control unit 24 inputs depth information of the fluorescent substance by the configuration processing (FIG. 2(2)) from the three-dimensional distribution calculation processing unit 30 as shown in FIG. 8. The data in Table 1 is read out, and the blocking range based on the depth information is determined (input). Based on the blocking range, the blocking control unit 24 controls the operation of the electric sliders 23A, 23B to move the blocking plates 42A, 42B to predetermined positions. As a result, about 80% of the fluorescence from the site where the fluorescent signal is the strongest in all the image ranges is blocked, and faint fluorescence can be detected in the second-time fluorescent image pickup (FIG. 2(4)). The third or later time fluorescent image pickup is conducted in the condition that all of the fluorescent images having the highest intensity in each of the former fluorescent image pickups are substantially blocked.

The operation in which the blocking mechanism 22 is the blocking mechanism 22B shown in FIG. 5 can also be controlled in the same manner.

When a living organ such as a lymph node, blood vessel, or heart is an object to be detected, the blocking mechanism 22B shown in FIG. 5 is suited. The computer 34 stores a correspondence table between the depth and the blocking range. The correspondence table is, for example, the one shown in Table 2, in which the blocking ranges with respect to the depths of a lymph node, blood vessel and heart are preliminarily determined because the depths (mm) from the sample surface viewed from the two-dimensional detector at which the lymph node, blood vessel, and heart are located are also known in advance. The correspondence table is also determined preliminarily by calculation so that the light attenuation of the fluorescence entering the two-dimensional detector 20 is one-fifth by the blocking.

TABLE 2

| | |
|---|---|
| 1 | Object to be blocked |
| 2 | Blocking range |
| 3 | Lymph node |
| 4 | Blood vessel |
| 5 | Heart |

As a precondition, the first-time image pickup of detecting a fluorescent image from the whole area of the object to be observed without using the blocking mechanism 22B has already been carried out.

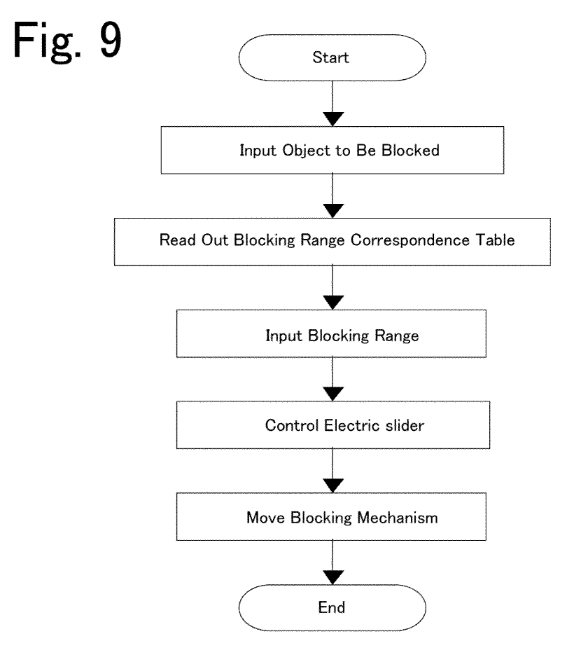

For the second-time fluorescent image pickup, since the organ having the highest fluorescent intensity has been found by the first-time image pickup, the organ is selected as an object to be blocked. As shown in FIG. 9, the blocking control unit 24 inputs what is an object to be blocked from the input unit 32. The blocking control unit 24 reads out data of Table 2, and determines (inputs) the blocking range for the object to be blocked. Based on the blocking range, the blocking control unit 24 controls the operation of the electric sliders 48A to 48D to move the blocking plates 46A to 46D to predetermined positions. As a result, the fluorescence from the organ showing the strongest fluorescent signal in all the image ranges is substantially blocked, and faint fluorescence from an organ other than the organ that is the object to be blocked can be detected in the second-time fluorescent image pickup (FIG. 2(4)). The third-time fluorescent image pickup is conducted in the condition that all of the fluorescent images having the highest intensity in each of the former fluorescent image pickups are substantially blocked.

### (Verification Result)

The present invention was verified by a cylindrical phantom, and the results are described below.

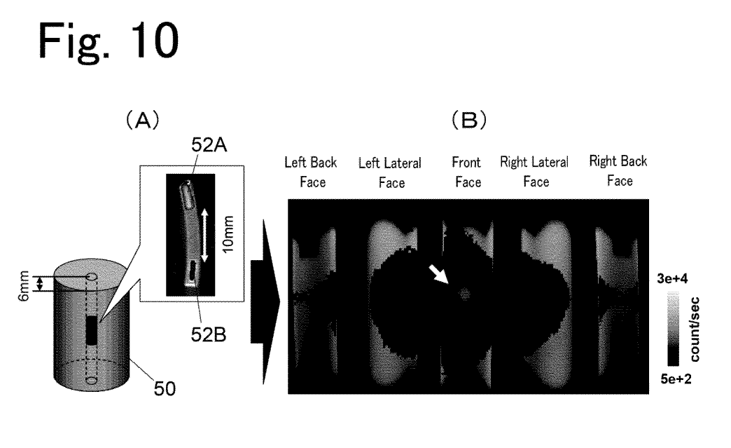

As shown in FIG. 10(A), a cylindrical phantom 50 (absorption coefficient 0.02 [/mm], scattering coefficient 10 [/mm]) having a diameter of 25 mm in which two tubes 52A and 52B containing ICG (indocyanine green) in concentrations that are different from each other by an order are installed was prepared. The tubes 52A, 52B were disposed at the positions of 6 mm depth from a certain circumferential face rather than at the center part of the cylinder of the phantom 50, and the distance between the tubes 52A, 52B was about 10 mm. The ICG concentration was 5 ppm for the tube 52A and 0.5 ppm for the tube 52B.

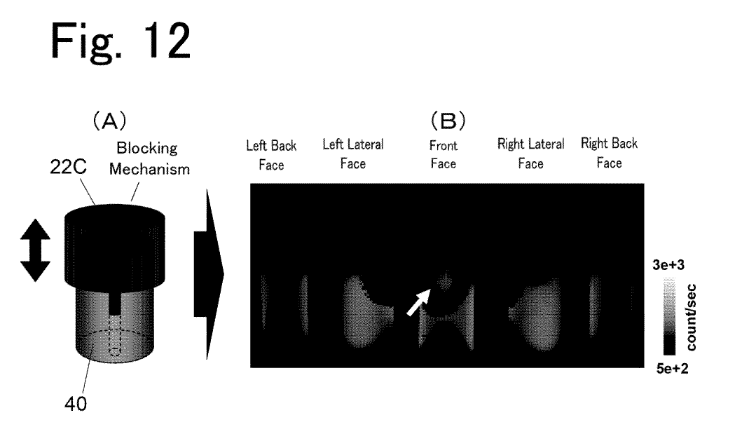

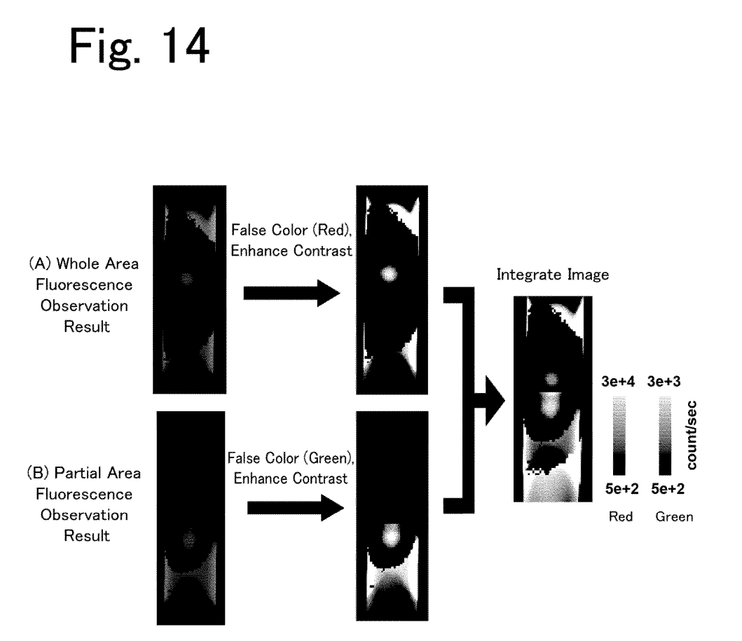

Under this condition, a fluorescent image of the whole area of the object to be observed was acquired, and fluorescence from the tube 52A was observed as shown in FIG. 10(B). Faint fluorescence from the tube 52B was also observed. A numerical value in FIG. 10(B) indicates fluorescence detection intensity, and for example, “5e+2” means “5×10²”. The same applies in FIG. 12 and FIG. 14.

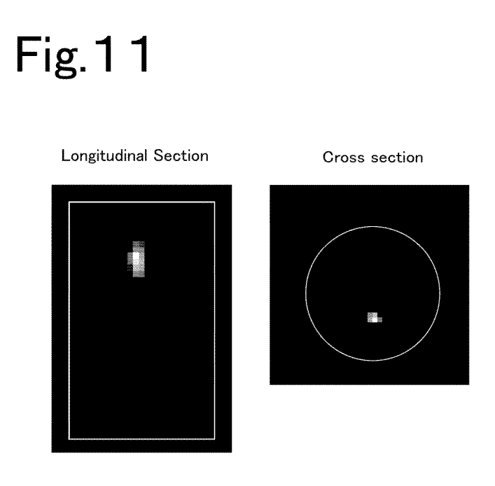

Next, internal information was calculated by using the fluorescent image data in FIG. 10(B), and a fluorescent image of the tube 52A was reconfigured at the position of 6 to 7 mm depth from the circumferential face of the two-dimensional detector side as shown in FIG. 11. In the calculation of this time, a fluorescent image of the tube 52B was also reconfigured in a concentration of about one-tenth because the concentration difference was only about one order.

Next, the blocking range of fluorescence was calculated from the preliminarily prepared table containing the correspondence between the positional information and the blocking range based on the living body internal information in FIG. 11. As the correspondence table, Table 1 was used. Since the fluorescent image from the tube 52A was reconfigured at the position of 6 to 7 mm depth from the phantom circumferential face on the two-dimensional detector side, 5.8 mm was employed as the blocking range corresponding to the depth of 7 mm from the data of Table 1.

Next, based on the information obtained from Table 1, the blocking mechanism 22C was actuated as shown in FIG. 12(A) to substantially block entry of the fluorescence from the tube 52A into the two-dimensional detector, and a fluorescent image was acquired again in that condition. The fluorescent image is shown in FIG. 12(B). In the result of FIG. 10(B), it was difficult to clearly detect a fluorescent image from the tube 52B due to the interference by the strong fluorescence from the tube 52A. However, by substantially blocking the fluorescence from the tube 52A, it was possible to increase the detection sensitivity of the two-dimensional detector, and the faint fluorescence from the tube 52B was detected with a favorable contrast as shown in FIG. 12(B) (the arrow in FIG. 12(B)).

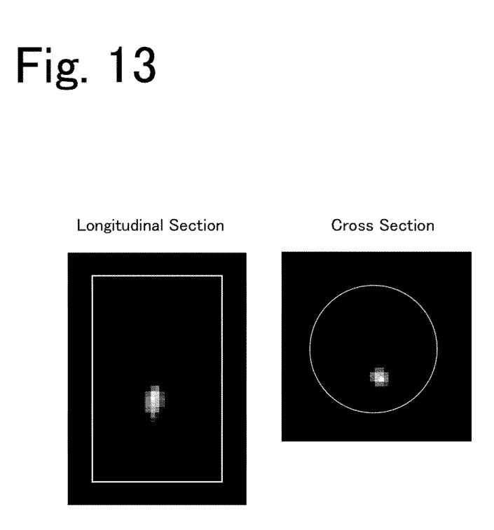

Next, internal information was calculated using fluorescent image data in FIG. 12(B), and a fluorescent image from the tube 52B was reconfigured at the position of 6 to 7 mm depth from the phantom circumferential face on the two-dimensional detector side as shown in FIG. 13.

Lastly, the fluorescence observation results obtained as in FIGS. 10 to 13 and the calculation results of living body internal information were integrated on the same image and displayed.

FIG. 14 shows fluorescent images obtained as in FIGS. 10 and 12 displayed on the same screen in two different colors. In displaying, the image was displayed after enhancing the contrast (intensities of less than or equal to a certain threshold were cut off) because the fluorescent image on the phantom surface spreads due to the influence of scattering.

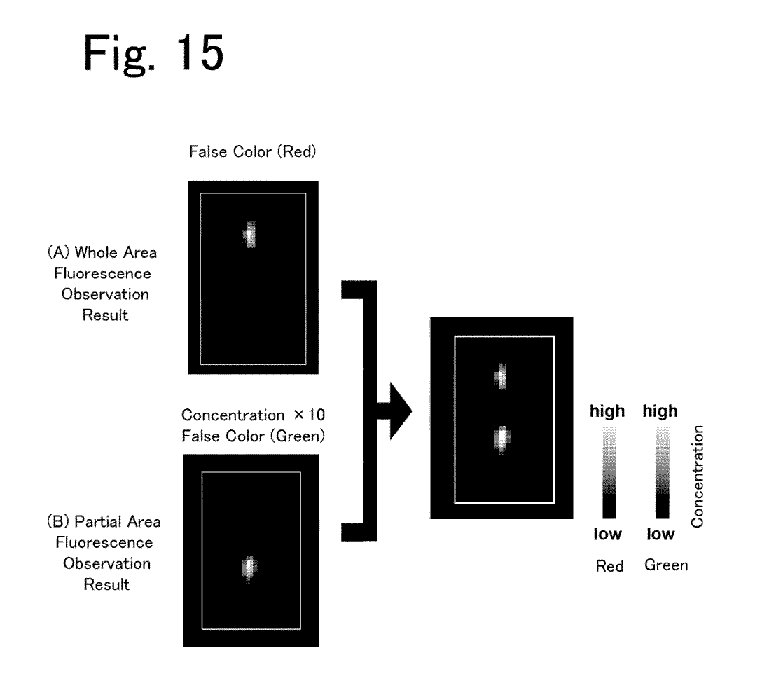

FIG. 15 shows the reconfigured fluorescence results obtained as in FIGS. 11 and 13 displayed on the same screen in two different colors. By using the reconfigured fluorescence results, it is possible to remove the noise by the excitation light or the like.

### DESCRIPTION OF REFERENCE SIGNS

10: Sample stage

12: Sample

14: Excitation light source

16: Excitation light

18: Image pickup unit

20: Two-dimensional detector

22, 22A, 22B, 22C: Blocking mechanism

24: Blocking control unit

26: Image integrating unit

28: Display unit

30: Three-dimensional distribution calculation processing unit

34: Computer

1-13. (canceled)

14. A fluorescent image acquisition device comprising:
a sample stage;
an excitation light source for irradiating a sample placed on the sample stage with excitation light which is a spreading light flux from a position distanced from the sample to excite a fluorescent substance in the sample to generate fluorescence;
an image pickup unit having a two-dimensional detector for detecting fluorescence from the sample;
a blocking mechanism disposed between the sample and the two-dimensional detector, for blocking entry of part of the fluorescence from the sample into the two-dimensional detector;
a blocking control unit for controlling the blocking mechanism in such a manner that entry of the strongest fluorescence from the sample into the two-dimensional detector is substantially blocked at a time when an image is to be taken by the image pickup unit;
an image integrating unit for calculating and processing in such a manner that at least two fluorescent images including at least one fluorescent image obtained by the image pickup unit in a condition that part of the sample is blocked by the blocking mechanism are integrated into one image; and
a display unit for displaying an integrated image obtained by the image integrating unit.

15. The fluorescent image acquisition device according to claim 14, further comprising:
a three-dimensional distribution calculation processing unit for calculating an interior of the sample from fluorescent image data obtained by the image pickup unit to reconfigure a fluorescent image,
wherein the image integrating unit is configured to integrate the fluorescent image reconfigured by the three-dimensional distribution calculation processing unit.

16. The fluorescent image acquisition device according to claim 15, wherein the blocking control unit controls the blocking mechanism in such a manner that entry of the strongest fluorescence into the two-dimensional detector is substantially blocked based on the fluorescent image reconfigured by the three-dimensional distribution calculation processing unit.

17. The fluorescent image acquisition device according to claim 14, further comprising:
an input unit for inputting internal information of a sample,
wherein the blocking control unit controls the blocking mechanism in such a manner that entry of the strongest fluorescence into the two-dimensional detector is substantially blocked based on sample internal information input from the input unit.

18. The fluorescent image acquisition device according to claim 14, wherein the blocking control unit controls the blocking mechanism in such a manner that a blocking range is varied depending on a depth of the fluorescent substance in the interior of the sample viewed from a direction of receiving fluorescence.

19. The fluorescent image acquisition device according to claim 14, wherein the blocking mechanism has a mechanism that automatically operates by electric control from the blocking control unit.

20. The fluorescent image acquisition device according to claim 14, wherein the display unit is configured to display an integrated image in different colors for different intensity ranges.

21. A method for acquiring a fluorescent image comprising the steps (A) to (D):
(A) a step of irradiating a sample with excitation light which is a spreading light flux from a position distanced from the sample to excite a fluorescent substance in the sample to generate fluorescence, and detecting the generated fluorescence by a two-dimensional detector to obtain a fluorescent image;
(B) at least one step of irradiating the sample with excitation light in a same manner as in the step (A) in a condition that entry of fluorescence from a part emitting the strongest fluorescence at a time when fluorescence is to be detected by the two-dimensional detector into the two-dimensional detector is substantially blocked, to obtain a fluorescent image by the two-dimensional detector;
(C) a step of integrating at least two fluorescent images including the fluorescent image obtained in the step (A) and at least the one fluorescent image obtained in the step (B) into one image; and
(D) a step of displaying the image integrated in the step (C).

22. The method for acquiring a fluorescent image according to claim 21, further comprising:
a reconfiguration step of calculating an interior of the sample from the fluorescent images obtained in the steps (A) and (B), and reconfiguring respective fluorescent images,
wherein in the image integrating step (C), the fluorescent images reconfigured in the reconfiguration step are integrated.

23. The method for acquiring a fluorescent image according to claim 22, wherein in the step (B), a part emitting the strongest fluorescence at the time when fluorescence is to be detected by the two-dimensional detector is determined based on the sample internal information obtained in the reconfiguration step.

24. The method for acquiring a fluorescent image according to claim 21, wherein in the step (B), a part emitting the strongest fluorescence at the time when fluorescence is to be detected by the two-dimensional detector is determined based on sample internal information input externally.

25. The method for acquiring a fluorescent image according to claim 21, wherein in the step (B), a blocking range is varied depending on a depth of the fluorescent substance in the interior of the sample viewed from a direction of receiving fluorescence.

26. The method for acquiring a fluorescent image according to claim 21, wherein in the step (D), an integrated image is displayed in different colors for different intensity ranges.
